(19) United States
(12) Patent Application Publication
(10) Pub. No.: US 2008/0247969 A1
(43) Pub. Date: Oct. 9, 2008

(54) Opaque multi-phase dentifrice with patterns

(76) Inventor: William Michael Glandorf, Mason, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/072,801

(22) Filed: Feb. 28, 2008

Related U.S. Application Data
(60) Provisional application No. 60/922,255, filed on Apr. 5, 2007.

(51) Int. Cl. A61K 8/02; A61Q 11/00

(52) U.S. Cl. 424/49

(57) ABSTRACT

A multi-phase dentifrice composition comprising at least two visually distinct phases wherein said visually distinct phases are packaged in a generally transparent container, wherein at least one phase is in physical contact with another phase, wherein all visually distinct phases are opaque, and wherein the visually distinct phases form any of a variety of patterns, excepting stripes.

### CROSS REFERENCE TO RELATED APPLICATIONS

This Application claims the benefit of U.S. Provisional Application No. 60/922,255, filed Apr. 5, 2007.

### FIELD OF THE INVENTION

The present invention relates to a multi-phased dentifrice composition comprising at least two visually distinct phases.

### BACKGROUND OF THE INVENTION

Aesthetics are known to play an important role in consumer choice and use of dentifrice. A unique visual appearance for a dentifrice provides an aesthetic effect that the user finds pleasing and promotes the use of the dentifrice.

In some cases, visual effects such as stripes or particles have been used to distinguish and market new dentifrice products. But there remains a continuous need for new and attractive visual variations for dentifrices. The present invention meets this need by providing a multi-phase dentifrice comprising at least two visually distinct phases. The visually distinct phases of this invention can be packaged to appear in many different patterns, shapes, and designs, resulting in appealing new visuals for dentifrice.

### SUMMARY OF THE INVENTION

The present invention is a multi-phase dentifrice composition comprising at least two visually distinct phases wherein said visually distinct phases are packaged in a generally transparent container, wherein at least one phase is in physical contact with another phase, wherein all visually distinct phases are opaque, and wherein the visually distinct phases form any of a variety of patterns, excepting stripes.

### BRIEF DESCRIPTION OF FIGURES

This patent or application file contains at least one photograph executed in color. Copies of this patent or patent application publication with color photographs will be provided by the Office upon request and payment of the necessary fee.

FIGS. 1-3 are three photographs, each photograph showing one embodiment of a multi-phase dentifrice in which all phases are opaque and form a pattern.

### DETAILED DESCRIPTION OF THE INVENTION

While the specification concludes with claims that particularly point out and distinctly claim the invention, it is believed the present invention will be better understood from the following description.

### DEFINITIONS

The term “comprising” as used herein means that other steps and other ingredients which do not affect the end result can be added. This term encompasses the terms “consisting of” and “consisting essentially of.” The compositions of the present invention can comprise, consist of, and consist essentially of the essential elements and limitations of the invention described herein, as well as any of the additional or optional ingredients, components, steps, or limitations described herein.

The term “effective amount” as used herein means an amount of a compound or composition sufficient to significantly induce a positive benefit, preferably an oral health benefit, but low enough to avoid serious side effects, i.e., to provide a reasonable benefit to risk ratio, within the sound judgment of a skilled artisan.

The term “oral composition” as used herein means a product that in the ordinary course of usage is not intentionally swallowed for purposes of systemic administration of particular therapeutic agents, but is rather retained in the oral cavity for a time sufficient to contact substantially all of the dental surfaces and/or oral tissues for purposes of oral activity. An oral composition may be in various forms including toothpaste, dentifrice, tooth gel, subgingival gel, foam, mouse, or denture product. An oral composition may also be incorporated onto strips or films for direct application or attachment to oral surfaces.

The term “dentifrice” as used herein means paste, gel, powder, or liquid formulations, unless otherwise specified, that are used to clean the surfaces of the oral cavity.

The term “teeth” as used herein refers to natural teeth as well as artificial teeth or dental prosthesis.

The term “polymer” as used herein shall include materials whether made by polymerization of one type of monomer or made by two (i.e., copolymers) or more types of monomers.

The term “water soluble” as used herein means that the material is soluble in water in the present composition. In general, the material should be soluble at 25° C. at a concentration of 0.1% by weight of the water solvent, preferably at 1%, more preferably at 5%, more preferably at 15%.

The term “phase” as used herein means a mechanically separate, homogeneous part of a heterogeneous system.

The term “multi-phase” as used herein means that at least two phases herein occupy separate but distinct physical spaces inside the container in which they are stored, but are in direct contact with one another.

The term “visually distinct” as used herein means a difference clearly perceived by sight.

The term “container” as used herein means a receptacle in which material is held or carried.

The term “opaque” as used herein means not transparent, generally transparent, or translucent; not allowing light to pass through.

The term “transparent” as used herein means capable of transmitting light so that objects or images are seen as if there was no intervening material.

The term “translucent” as used herein means that light is diffused as it passes through so that objects or images are seen, but without clarity.

The term “generally transparent container” as used herein means that at least some of the container is capable of being seen through so that the appearance of the container's contents may be visualized. The term includes transparent and translucent containers, wherein contents in a transparent container can be more clearly visualized than those in a translucent container. For purposes of the invention, as long as one wavelength in the visible light range has greater than 25% transmittance, it is considered to be generally transparent.

The term “packaged” as used herein means to be placed and held inside of.

The term “packaging layer” as used herein means any further bundling or wrapping of the dentifrice composition beyond the container, including but not limited to a label, shrink wrap, stretch wrap, or a box.

The term “label” as used herein means any decoration or information that is attached or made part of a container.

The term “shrink wrap” as used herein means to wrap and seal in a flexible film of plastic.

The term “pattern” as used herein means a decorative or distinctive design, not necessarily repeating or imitative, including but not limited to the following: marbled, check, mottled, veined, clustered, geometric, spotted, helical, swirl, arrayed, variegated, textured, spiral, cycle, contoured, laced, tessellated, starburst, lobed, lightning, blocks, textured, pleated, cupped, concave, convex, braided, tapered, and combinations thereof.

The term “band” as used herein means a continuous stroke that can be straight (i.e., without bend, angle, or curve) or non-straight (e.g., curved, angled, or wavy) and that can vary in thickness throughout.

The term “stripes” as used herein means alternating bands that run without bend, angle, or curve.

The term “alternating” as used herein means to interchange repeatedly.

The term “physical contact” as used herein means touching yet not mixing.

The term “petals” as used herein means the appearance of loose floral (e.g. roses) petals layered on top of one another.

The term “spiral” as used herein means the appearance of a helix or the appearance of a curve generated by a point moving around a fixed point while constantly receding from or approaching it.

The term “marbled” as used herein means a mottled or variegated appearance that could include swirls, spots, or blotches of different colors or shades.

The term “swirl” as used herein means the appearance of a curve.

The term “geometric” as used herein means an appearance resembling or employing the simple rectilinear or curvilinear lines or figures used in geometry.

The term “starburst” as used herein means a shape or design with emanating rays.

The term “lightning” as used herein means a pattern or shape of lightning, that is, a pattern of jagged streaks.

The term “blocks” as used herein means a series of segments laid end-to-end, each segment being generally shaped as a square or rectangular. Each segment appears visually distinct from the segment preceding it, but the same visually distinct segment may appear more than once.

The term “benefit phase” as used herein means that a particular phase of the composition provides a desired effect, including but not limited to whitening, long-lasting refreshment, flavor, clean feeling, improved health benefits, improved efficacy, and combinations thereof.

The term “dispense” or “dispensing” as used herein means to administer or remove.

The term “dispenser” as used herein means any pump, tube, package, or container suitable for dispensing oral compositions.

The term “designated volume ratio” as used herein means fixed proportional amounts of material.

The term “longitudinal axis” as used herein means the longest axis of a body.

The term “non-intersecting bands” as used herein means bands that do not cut across or through each other and that do not narrow and merge.

The term “intersect” as used herein means bands that cut across or through each other, or that narrow and merge.

The term “oriented” as used herein means aligned or positioned.

The term “direction” as used herein means course or bearing.

The term “parallel” as used herein means extending in the same direction and having common perpendiculars.

The term “adjacent” as used herein means adjoining or neighboring.

The term “thickness” as used herein means the width of a band of a single phase.

The term “irregular interface” as used herein means the surface regarded as the common boundary of two phases is jagged or some other nonlinear alignment.

The term “wavy” as used herein means curving alternately in opposite directions.

The term “character” as used herein means an image that includes but is not limited to letters, numerals, symbols, emblems, figures, signs, images, marks, logos, trademarks, depictions, shapes, and monograms.

The term “symbol” as used herein means an image used to represent something.

The term “letter” as used herein means a symbol used to represent a speech sound and that is part of an alphabet.

The term “numeral” as used herein means a symbol expressing a number.

The term “emblem” as used herein means a sign, design, or figure that identifies or represents something.

The term “figure” as used herein means a form or shape as determined by outlines.

The term “design” as used herein means an ornamental pattern or scheme.

The term “ribbons” as used herein means the appearance created by a narrow strip or band of one phase of material layered with a narrow strip or band of one or more other phases.

The term “colored” as used herein means having color.

The term “tinted” as used herein means a shade of a color, especially a pale or delicate variation.

The term “shaded” as used herein means the degree of darkness of a color, determined by the quantity of black or by the lack of illumination.

The term “frosted” as used herein means a decoration or coating added to a surface.

The term “pearlescent” as used herein means having an iridescent luster, resembling that of a pearl.

The term “photosensitive” as used herein means sensitive to light or similar radiation.

The term “equidistant” as used herein means the same distance apart at every point.

The term “fully disposed” as used herein means that two phases are coaxial, with one phase fully enclosing the other.

The term “coil” as used herein means a series of spirals or rings.

The term “continuous” as used herein means that, during the filling of the container, the filling procedure of a particular phase into the container is not stopped.

The term “discontinuous” as used herein means that, during the filling of the container, the filling procedure of a particular phase into the container is stopped at least once, either by random stops and starts or with regular, or cyclic, stops and starts.

The term “textured” as used herein means having surface roughness.

The term “pleated” as used herein means a folded appearance.

The term “cupped” as used herein means the edges are curved.

The term “concave” as used herein describes a surface or boundary that curves inward.

The term “convex” as used herein means having a surface or boundary that is curved or rounded outward.

The term “braided” as used herein means the appearance of being interweaved.

The term “tapered” as used herein means to become gradually thinner or narrower toward one end.

The term “piled” as used herein means an assemblage of things laid or lying one upon the other.

The term “overlap” as used herein means to cover over a part of, or to have an area in common.

The term “intertwining” as used herein means to spin or twist together.

The term “cylindrical” as used herein means having the shape of a cylinder, that is, a tube with a consistent cross-sectional area and two equally-sized circular ends.

The term “non-cylindrical” as used herein means any and all shapes that are not a tube with a consistent cross-sectional area and two equally-sized circular ends.

The term “through” as used herein means in at one end, side, or surface and out at the other.

The term “translating” as used herein means a motion without rotation or angular displacement.

The term “oscillating” as used herein means to swing or move to and fro, like a pendulum.

The term “reciprocating” as used herein means motion alternately backward and forward.

The term “vibrating” as used herein means to move to and fro or up and down quickly and repeatedly.

The term “pulsating” as used herein means to expand and contract rhythmically.

The term “rotating” as used herein means to turn around an axis or center point.

The term “plunging” as used herein means to cast or thrust into something.

All percentages, parts and ratios are based upon the total weight of the compositions of the present invention, unless otherwise specified. All such weights as they pertain to listed ingredients are based on the active level and, therefore, do not include solvents or by-products that may be included in commercially available materials, unless otherwise specified. The term “weight percent” may be denoted as “wt. %” herein.

All molecular weights as used herein are weight average molecular weights expressed as grams/mole, unless otherwise specified.

### Embodiments

The present invention is directed to a multi-phase dentifrice composition comprising at least two visually distinct phases, wherein said visually distinct phases are packaged in a generally transparent container, at least one phase is in physical contact with another phase, and the phases form a unique visual appearance.

It is understood that the visual appearances described herein are of the composition as it is in the container. That is, the descriptions depict the combined appearance of the composition, the container, and any further packaging layer, not just the composition alone or of the composition as dispensed from the container.

In some embodiments, the visually distinct phases form any of a variety of patterns, excepting stripes. The patterns that may be formed include, but are not limited to, swirls, spirals, marbled, geometric, petals, starburst, lightning, blocks, and combinations thereof. Patterns may appear two-dimensional or three-dimensional, depending on whether the phases are opaque or transparent; as long as at least one phase is generally transparent, the pattern appears three-dimensional. Some embodiments may have more than one pattern.

In other embodiments, the visually distinct phases may form the appearance of at least one character, where the characters include, but are not limited to, letters, numerals, symbols, emblems, figures, and combinations thereof.

In some embodiments, at least one visually distinct phase may form a coil through at least one other visually distinct phase. In some embodiments, particularly embodiments that appear three-dimensional, there is a primary pattern and a secondary pattern. The primary pattern may be a coil, while the secondary pattern may be the shape or texture of the coil itself.

In any coil embodiment, a coil may be continuous. As used herein, “continuous” means that the phase is literally connected from one end of the container to the other. But a coil, either continuous or discontinuous, may not necessarily appear connected, and it may not necessarily appear centered. Such a coil may be uniform, meaning that it is regularly spaced, or it may be non-uniform, meaning irregularly spaced. A coil may be at an angle within the container, or it may be along the container's longitudinal axis. A coil may touch the container or may be entirely enclosed within another phase or phases.

A coil may be compacted or compressed, or it may be stretched out. As the degree of compression, i.e., the slope or pitch of the coil, varies, the coil's appearance is affected. For example, if compacted, a coil may overlap itself and appear rippled or mounded, as if it was loosely piled as it continuously fell. An overlapping coil may appear like a coiled rope, piles, or as seaweed. An overlapping coil may appear like a compacted spring, or appear interwoven. Though in some embodiments, one continuous phase may not actually be a coil, but may still overlap itself and appear rippled or mounded like seaweed or a coiled rope.

Alternatively, a compacted coil may look like petals or leaves that are layered, stacked, or piled. A compacted coil may also appear as alternating flaps that are draped, nested, or interlaced with one another. As a coil is less compacted, or stretched out, it may appear more like a helix and be more uniform.

The secondary pattern may reflect the shape or texture of a coil itself. A coil may be cupped, concave, or convex, having a scooped-out appearance. A coil may appear braided, checked, or interwoven, or it may appear tapered. Alternatively, a coil may appear textured or pleated. The variables of the secondary pattern may be independent from the variables of the primary pattern. That is, the shape and texture of the coil may not necessarily be affected by the degree of compression or the uniformity of the coil within another phase or phases.

For all the embodiments described where a phase forms a coil through another phase, there may be more than one coil appearing through one or more other phases. In some embodiments, there may be more than one coil formed by a single visually distinct phase that appear through one or more other phase. In some coil embodiments, the container may be non-cylindrical. In some coil embodiments, the total volume of all coil phases may be more than about 10% of the volume of all phases combined. In other coil embodiments, the total volume of all coil phases may be more than about 12% of the volume of all phases combined. In other coil embodiments, the total volume of all coil phases may be more than about 15% of the volume of all phases combined. In other coil embodiments, the total volume of all coil phases may be more than about 20% of the volume of all phases combined. In still other coil embodiments, the total volume of all coil phases may be more than about 30% of the volume of all phases combined.

In some coil embodiments, all phases may be coils, that is, the total volume of all coil phases is the volume of all phases combined. In some embodiments in which all phases are coils, all the coils may intertwine throughout the container. In some embodiments, each intertwining coil may have a constant thickness and all intertwining coils may have about the same thickness. In other embodiments, the thickness of the coils may vary from each other, or the thickness of any particular coil may vary throughout. In other embodiments, the thickness of one intertwining coil may be at least two times the thickness of another intertwining coil. In some embodiments, the intertwining coils may have an irregular interface.

As with the pattern embodiments, coil embodiments may appear two-dimensional or three-dimensional, depending on whether the phases are opaque or transparent; as long as at least one phase is generally transparent, the composition's appearance is three-dimensional. When all the phases are opaque, the product's appearance may still be described as a coil through another phase or phases, or as intertwining coils. There may be a secondary pattern reflecting the shape or texture of the coil itself, at least one coil may overlap itself, or the container may be non-cylindrical. In some all-opaque embodiments, the total volume of all coil phases may be more than 10% of the volume of all phases combined.

But one opaque phase forming a coil through another opaque phase or phases may also be described in two dimensions. For example, some embodiments may resemble the appearance of a candy cane or a barber's pole.

Alternatively, in the embodiments where all phases are opaque, the visually distinct phases may appear and may be described not only as patterns or coils, but also as bands. In this context, a band is understood to be a continuous stroke of one phase that can be straight or non-straight and that can vary in width throughout.

For example, in some embodiments where all phases are opaque, the visually distinct phases form alternating bands where at least one band is oriented in a direction not parallel to the longitudinal axis of the container. In other embodiments where all phases are opaque, the visually distinct phases form alternating bands where at least one band is oriented in a direction not parallel to the direction that the composition is dispensed from the container.

Embodiments in which at least one phase is generally transparent may also be described as alternating bands, wherein at least one band is oriented in a direction not parallel to the longitudinal axis of the container or in a direction not parallel to the direction that the composition is dispensed from the container.

In any embodiment described as having alternating bands, the alternating bands may be non-intersecting or there may be at least one band that intersects with an adjacent band. In some alternating band embodiments, any two adjacent bands may be generally parallel. In other embodiments, each band may have a constant thickness while all bands have about the same thickness. In other embodiments, the thickness of the bands of one visually distinct phase may be at least two times the thickness of the bands of another visually distinct phase.

In still other embodiments with alternating bands, the alternating bands may have an irregular interface. For example, the interface may be jagged or some other nonlinear alignment. In other embodiments, the bands of one phase may appear patterned, such as being textured, pleated, cupped, concave, convex, braided, or tapered. And for any embodiment with alternating bands, the container may be non-cylindrical.

In still other embodiments of the present invention, the combination of the dentifrice composition plus the container may create the appearance of a pattern. In other embodiments, the combination of the dentifrice composition, the container, and at least one packaging layer may form a pattern. A packaging layer is any further bundling or wrapping of the dentifrice composition beyond the container, including but not limited to a label, shrink wrap, stretch wrap, or a box. In still other embodiments, the combination of the dentifrice composition and at least one packaging layer may create the appearance of a pattern.

In any embodiment in which the container and/or a packaging layer help form the unique appearance, the dentifrice composition may be multi-phased where each phase is visually distinct, or the dentifrice composition may be a single phase.

In the embodiments in which the container and/or packaging layer help form a pattern, the patterns that may be formed include but are not limited to stripes, marbled, spiral, geometric, starburst, lightning, blocks, and combinations thereof. In embodiments in which the container and/or packaging layer help form a pattern, the container or packaging layer appearance may be striped, colored, tinted, shaded, frosted, or patterned.

In any embodiment of the dentifrice composition, at least one visually distinct phase may comprise a benefit phase. In some embodiments, the visually distinct phases may appear to be randomly oriented.

For any particular embodiment described above, additional factors may create varied appearances. A particular embodiment, i.e., a described pattern, coil, or band formation, may encompass numerous appearances due to additional factors that include, but are not limited to, the appearance of a phase, container or packaging layer effects, the filling procedure, the motion or motions of a filling nozzle or nozzles, motion of the container while filling, effects achieved after filling, or the orientation of the product in the container.

For example, the appearance of a phase may be varied by its color, its width or thickness as a coil or band, transparency vs. opacity, pearlescence, texture, photosensitivity, or by suspended particles in the phase. The appearance of a phase may be patterned, such as being pleated, cupped, concave, convex, braided, tapered, or textured. In any embodiment, each visually distinct phase may comprise at least about 10% of the volume of all phases combined.

Container or packaging layer effects that may also create varied appearances of a particular embodiment include, but are not limited to, colors, shades, tints, frosting, patterns, stripes, transparency, translucency, shapes, holography, labels, shrink wrap, stretch wrap, optical illusions, logos, characters, and particles. Another container effect may be a strip down the center of the container, which may or may not contact the dentifrice composition. Such a strip may have printing or a design on it. Still another container effect may be printing on the inside of the container in soluble ink that interacts with the dentifrice composition. Any of these container and/or packaging layer effects may create any of the visual appearances described herein.

The visually distinct phases may be packaged in a generally transparent container. In one aspect, at least 5%, 10%, 20%, 30%, 40%, 50%, 60%, 70%, 80%, 90%, or even 100% of the container's surface area may be generally transparent. Materials from which said generally transparent portion may be made include, but are not limited to: polypropylene (PP), polyethylene (PE), polycarbonate (PC), polyamides (PA), polyethylene terephthalate (PETE), polyvinylchloride (PVC), general purpose polystyrene (GPPS), and polystyrene (PS). The generally transparent portion of said container may have a transmittance of more than 25%, 30%, 40%, 50%, 60% or even more than 70% in the visible part of the spectrum (approx. 410-800 nm). For purposes of the invention, as long as one wavelength in the visible light range has greater than 25% transmittance, it is considered to be generally transparent.

A portion of the container or the entire container may be tinted, shaded, colored, frosted, patterned, or striped. Such container appearances may be achieved, for example, by including colorant in the resin during manufacture of the container. The appearances may also be attained by adding decorations to a finished container, or by printing on, embossing, or stamping an already-manufactured container. Shrink-wrapping or stretch-wrapping the container or portion of the container may also create the described appearances for the container. In addition, any combination of the described methods could be used to create various container appearances. Unique visual appearances may be created by the visually distinct phases alone, by the container, or by a combination of the visually distinct phases and the container.

The pattern created by the visually distinct phases, the container, or a combination of the visually distinct phases and the container may be laser-activated, meaning that a photosensitive substance is included in at least one of the visually distinct phases or the container and then targeted with a laser to produce a discrete pattern.

The container of the present invention may be of any form, shape, or size suitable for storing and packaging dentifrice. Examples of forms include tubes, bottles, tottles, thermoforms, or pouches. The shape of the container may be, for example, cylindrical, which is defined as a tube with a consistent cross-sectional area and two equally-sized circles on either end. Any container shape that does not have two equally-sized circles on the ends is non-cylindrical. For example, the container may be oval-shaped at the ends, wherein the two ovals may be the same size or different sizes, and the body of the container has a generally oval-shaped cross-section at all points. The shape of the container may affect the visual appearance of the phases, for example, by affecting the colors or by creating the appearance of layers. The size of the container may range from a single dose up to 30 oz. (860 grams), preferably up to 20 oz. (570 grams), and more preferably up to 14 oz. (400 grams). Ways that the phases may be dispensed from the container include, for example, squeezing the container, by a pump mechanism, or by gravity.

The container that the visually distinct phases are packaged in may have a label adhered to it. The label may be transparent, generally transparent, or opaque. The label may be colored, shaded, tinted, patterned, or striped. The label may be in any shape, including simple shapes such as bands, squares, rectangles, rectangles with round corners, circles, or ovals, or more complicated shapes, for example, shapes such as letters. The label may cover up to 100% of the container. The label may contain multiple pages. The label may be printed inside out so as to be read through a transparent product. All or part of the label may be shrink-wrapped or stretch-wrapped onto the container. Labeling of the container may be etched into the mold of the container or embossed on the container, and, in some embodiments, then printed on. Unique visual appearances may be created by the visually distinct phases alone, by the label appearance, or by a combination of the visually distinct phases and the label.

Any packaging layer, such as shrink wrap, stretch wrap, or a box, for the dentifrice composition may be patterned, colored, shaded, tinted, or striped.

The filling procedure of the phases into the container may be done continuously at a steady rate, done continuously at varying rates, or may be done discontinuously with random stops and starts or with regular, or cyclic, stops and starts. Motions of the nozzle, nozzles, or the container while filling include, but are not limited to, oscillating, reciprocating, translating, vibrating, pulsating, rotating, and plunging. Effects achieved after filling include, but are not limited to, centrifuging, shaking, changing temperature, changing pressure, adding or removing air, using electromagnetic radiation, and using sonic energy.

Multiple filling nozzles may be used to achieve the described visual appearances. Nozzle diameters may range from 1/16 inch (1.5875 mm) up to the size of the opening of the container, but preferably range from ¼ inch to 1 inch (6.35 mm to 25.4 mm). The arrangement of filling nozzles may be concentric or side-by-side. Concentric nozzles may be flush or protruding.

Achieving the visual appearances described herein may be accomplished with modifications to standard, high-viscosity filling equipment, for example tube fillers from IWK or Norden, or with other fill systems, such as modifications to standard liquids fillers, for example with fillers sold by Pneumatic Scale, Krones, or Ronchi.

The dosing process for the desired appearance is achieved through controlled dosing through a filling nozzle of each phase of the dentifrice, for example with a stepper motor, servo motor, mass flow meter, magnetic flow meter, or metering pump. The dosing of each phase may be coordinated through mechanical or electrical synchronization of the flows. Different phases may be injected into the filling nozzle through nozzle segmentation, such as on standard multi-color striping dentifrice machines from IWK or Norden, or with secondary flows injected at various locations in the filling nozzle.

The container being filled may be cylindrical, for example a standard dentifrice tube, or a different shape such as a bottle, which may be designed to stand or rest on its base or its closure.

Filling of the container may involve relative motion between the container and the filling nozzle, suitably accomplished by moving the container while holding the nozzle fixed, by moving the nozzle while holding the container fixed, or by moving both the filling nozzle and the container simultaneously.

The relative motion of the filling nozzle and container may involve any controlled combination of rotational, vertical, horizontal, or orbital-oscillating or non-oscillating motion. This motion would suitably be accomplished by mechanical or electrical synchronization of the dosing and relative motions through devices such as mechanical line-shafts and cams, or electrical stepper or servo motors.

A filling nozzle suitable for filling the visually distinct phases into a container is described in WO 2006/125663, which is incorporated by reference herein. Such a filling nozzle comprises a tubular body having an internal tubular primary conduit for flow of a primary phase, bounded by a peripheral wall, adapted for the introduction of a primary phase at an upstream position of the conduit, having a downstream end adapted to be inserted into a container to be filled, an outlet opening at a downstream end of the conduit via which a phase may flow from the conduit into a container, within the conduit at least one secondary conduit for the flow of a secondary phase, adapted for the introduction of the secondary phase at an upstream part of the secondary conduit, the secondary conduit having at least one outlet nozzle adjacent a downstream end of the secondary conduit configured to introduce a stream of the secondary phase into a flow of the primary phase in the primary component.

To complete the filling of the visually distinct phases into a container, an apparatus may be used, as further described in WO 2006/125663, comprising the filling nozzle described above, a support for the container, means to move the support and filling nozzle relatively toward each other so that the downstream end of the filling nozzle may be inserted into the container, means to introduce primary and secondary phases into the respective primary and secondary conduits such that the phases flow out of the outlet opening of the filling nozzle relatively apart as the visually distinct phases flow into the container, and means to cause relative rotation of the filling nozzle and container about the upstream-downstream axis as the filling nozzle and container move relatively apart.

A process for filling a container with visually distinct phases comprises the steps of providing an apparatus as described above, providing a container, moving the container and filling nozzle relatively toward each other so that the downstream end of the filling nozzle becomes inserted into the container, introducing primary and secondary phases into the respective primary and secondary conduits such that the phases flow out of the outlet opening of the filling nozzle into the container, moving the container and filling nozzle relatively apart as the phase flows into the container, and relatively rotating the filling nozzle and container about the upstream-downstream axis as the filling nozzle and container move relatively apart, to thereby form a number of unique appearances.

Alternatively, various filling nozzle assemblies and filling apparatuses are described in U.S. Pat. No. 6,516,838, U.S. Pat. No. 6,245,344, U.S. Pat. No. 6,367,519, and U.S. Pat. No. 6,213,166, which are incorporated by reference herein. The visually distinct phases may be filled into a container by a filling apparatus comprising a nozzle assembly having at least two nozzles coupled together in close configuration, at least two pumps for pumping each of the phases stored in separate storage bins each interconnected by a suction hose to each pump, at least two hoses interconnected to the nozzles and the pumps, a support and alignment funnel coupled to the apparatus for supporting the container to be filled in an upright position, a drive motor coupled to the nozzle assembly adapted to rotate the nozzle assemble and move the nozzle assembly in a vertical direction during filling of the container, and a base located adjacent to the support and alignment funnel.

One process for filling a container with visually distinct phases comprises the steps of providing at least two visually distinct phases, arranged in separate storage bins each having a pump and a hose attached thereto, moving a container for receiving a resulting product formed by the at least two visually distinct phases into position relative to a support and alignment funnel, pumping the at least two visually distinct phases through the respective hoses into a nozzle assembly having at least two nozzles for filling the container, rotating the nozzle assembly, and combining predetermined amounts of each of the at least two visually distinct phases for creating the resulting product housed in a single container, wherein the resulting product has the at least two visually distinct phases form a unique appearance.

Another process for filling a container with the visually distinct phases comprises the steps of providing a filling apparatus as described above, mounting the container on the base, signaling a commencement step from the filling apparatus, placing the nozzle assembly directly over the container and the support and alignment funnel, dropping the nozzle assembly into the container whereby the tip of the nozzles are proximate to a bottom portion of the container, providing relative rotational movement between the nozzle and the container at a predetermined number of revolutions per minute, starting the at least two pumps, providing relative vertical movement causing increased separation between the nozzle assembly and a bottom of the container, controlling a rate of flow of each of the phases by the pumps, and urging the phases through the respective hoses to fill the container.

### Dentifrice Compositions

The dentifrice compositions of the present invention may be typical dentifrice formulations. Each of the multi-phases may be a separate composition or may be generally the same except for something that makes it visually distinguishable. The material that changes the visual appearance of a phase may be added at the very end of production so that the two or more compositions can be formed in one batch and then differentiated at the last point in the process before or as filling occurs. The material added to distinguish a phase may be a colorant, dye, titanium dioxide, opacifying agent, brightening agent, pearlescent, photosensitive material, or a type of particle. The actual material added may be visible itself or it may cause an effect that is visible in the final composition. A material itself may be the separate phase. For example, during filling, a layer of sparkles may be added that is visible. This would create a visually distinct phase. Each of the visually distinct phases may have the same viscosity or different viscosities.

Dentifrice compositions are well known. The selection of a particular composition will depend on the visual appearance desired and on secondary considerations like taste, cost, stability, benefits desired, etc. The following includes examples of suitable materials in dentifrice compositions.

The dentifrice composition may comprise suitable cosmetic and/or therapeutic actives. Such actives include any material that is generally considered safe for use in the oral cavity and that provides changes to the overall appearance and/or health of the oral cavity, including, but not limited to, anti-calculus agents, fluoride ion sources, stannous ion sources, whitening agents, anti-microbial, anti-plaque agents, anti-inflammatory agents, nutrients, antioxidants, anti-viral agents, analgesic and anesthetic agents, H-2 antagonists, and mixtures thereof. When present, the level of cosmetic and/or therapeutic active in the oral composition is, in one embodiment from about 0.001% to about 90%, in another embodiment from about 0.01% to about 50%, and in another embodiment from about 0.1% to about 30%, by weight of the oral composition.

The following is a non-limiting list of actives that may be used in the present invention.
a) Fluoride Ion

The present invention may comprise a safe and effective amount of a fluoride compound (e.g. water soluble). The fluoride ion may be present in an amount sufficient to give a fluoride ion concentration in the composition at 25° C., and/or in one embodiment can be used at levels of from about 0.0025% to about 5.0% by weight, in another embodiment from about 0.005% to about 2.0% by weight, to provide anticaries effectiveness. A wide variety of fluoride ion-yielding materials can be employed as sources of soluble fluoride in the present compositions. Examples of suitable fluoride ion-yielding materials are disclosed in U.S. Pat. Nos. 3,535,421, and 3,678,154. Representative fluoride ion sources include: stannous fluoride, sodium fluoride, potassium fluoride, amine fluoride, sodium monofluorophosphate and many others. In one embodiment the dentifrice composition comprises stannous fluoride or sodium fluoride, as well as mixtures thereof.
b) Anticalculus Agent

Dentifrice compositions of the present invention may also comprise an anti-calculus agent, which in one embodiment may be present from about 0.05% to about 50%, by weight of the dentifrice composition, in another embodiment is from about 0.05% to about 25%, and in another embodiment is from about 0.1% to about 15%. The anti-calculus agent may be selected from the group consisting of polyphosphates (including pyrophosphates) and salts thereof; polyamino propane sulfonic acid (AMPS) and salts thereof; polyolefin sulfonates and salts thereof; polyvinyl phosphates and salts thereof; polyolefin phosphates and salts thereof; diphosphonates and salts thereof; phosphonoalkane carboxylic acid and salts thereof; polyphosphonates and salts thereof; polyvinyl phosphonates and salts thereof; polyolefin phosphonates and salts thereof; polypeptides; and mixtures thereof. In one embodiment, the salts are alkali metal salts. Polyphosphates are generally employed as their wholly or partially neutralized water-soluble alkali metal salts such as potassium, sodium, ammonium salts, and mixtures thereof. The inorganic polyphosphate salts include alkali metal (e.g. sodium) tripolyphosphate, tetrapolyphosphate, dialkyl metal (e.g. disodium) diacid, trialkyl metal (e.g. trisodium) monoacid, potassium hydrogen phosphate, sodium hydrogen phosphate, and alkali metal (e.g. sodium) hexametaphosphate, and mixtures thereof. Polyphosphates larger than tetrapolyphosphate usually occur as amorphous glassy materials. In one embodiment the polyphosphates are those manufactured by FMC Corporation, which are commercially known as Sodaphos (n≈6), Hexaphos (n≈13), and Glass H (n≈21, sodium hexametaphosphate), and mixtures thereof. The pyrophosphate salts useful in the present invention include, alkali metal pyrophosphates, di-, tri-, and mono-potassium or sodium pyrophosphates, dialkali metal pyrophosphate salts, tetraalkali metal pyrophosphate salts, and mixtures thereof. In one embodiment the pyrophosphate salt is selected from the group consisting of trisodium pyrophosphate, disodium dihydrogen pyrophosphate (Na₂H₂P₂O₇), dipotassium pyrophosphate, tetrasodium pyrophosphate (Na₄P₂O₇), tetrapotassium pyrophosphate (K₄P₂O₇), and mixtures thereof. Polyolefin sulfonates include those wherein the olefin group contains 2 or more carbon atoms, and salts thereof. Polyolefin phosphonates include those wherein the olefin group contains 2 or more carbon atoms. Polyvinylphosphonates include polyvinylphosphonic acid. Diphosphonates and salts thereof include azocycloalkane-2,2-diphosphonic acids and salts thereof, ions of azocycloalkane-2,2-diphosphonic acids and salts thereof, azacyclohexane-2,2-diphosphonic acid, azacyclopentane-2,2-diphosphonic acid, N-methyl-azacyclopentane-2,3-diphosphonic acid, EHDP (ethane-1-hydroxy-1,1,-diphosphonic acid), AHP (azacycloheptane-2,2-diphosphonic acid), ethane-1-amino-1,1-diphosphonate, dichloromethane-diphosphonate, etc. Phosphonoalkane carboxylic acid or their alkali metal salts include PPTA (phosphonopropane tricarboxylic acid), PBTA (phosphonobutane-1,2,4-tricarboxylic acid), each as acid or alkali metal salts. Polyolefin phosphates include those wherein the olefin group contains 2 or more carbon atoms. Polypeptides include polyaspartic and polyglutamic acids.
c) Stannous Ion

The dentifrice compositions of the present invention may include a stannous ion source. The stannous ions may be provided from stannous fluoride and/or other stannous salts. Stannous fluoride has been found to help in the reduction of gingivitis, plaque, sensitivity, and in improved breath benefits. The stannous ions provided in a dentifrice composition will provide efficacy to a subject using the dentifrice composition. Although efficacy could include benefits other than the reduction in gingivitis, efficacy is defined as a noticeable amount of reduction in in situ plaque metabolism. Formulations providing such efficacy typically include stannous levels provided by stannous fluoride and/or other stannous salts ranging from about 3,000 ppm to about 15,000 ppm stannous ions in the total dentifrice composition. The stannous ion is present in an amount of from about 4,000 ppm to about 12,000 ppm, in one embodiment from about 5,000 ppm to about 10,000 ppm. Other stannous salts include organic stannous carboxylates, such as stannous acetate, stannous gluconate, stannous oxalate, stannous malonate, stannous citrate, stannous ethylene glycoxide, stannous formate, stannous sulfate, stannous lactate, stannous tartrate, and the like. Other stannous ion sources include, stannous halides such as stannous chlorides, stannous bromide, stannous iodide and stannous chloride dihydride. In one embodiment the stannous ion source is stannous fluoride in another embodiment, stannous chloride dihydrate. The combined stannous salts may be present in an amount of from about 0.001% to about 11%, by weight of the dentifrice compositions. The stannous salts may, in one embodiment, be present in an amount of from about 0.01% to about 7%, in another embodiment from about 0.1% to about 5%, and in another embodiment from about 1.5% to about 3%, by weight of the dentifrice composition.
d) Whitening Agent

A whitening agent may be included as an active in the present dentifrice compositions. The actives suitable for whitening are selected from the group consisting of alkali metal and alkaline earth metal peroxides, metal chlorites, perborates inclusive of mono and tetrahydrates, perphoshates, percarbonates, peroxyacids, and persulfates, such as ammonium, potassium, sodium and lithium persulfates, and combinations thereof. Suitable peroxide compounds include hydrogen peroxide, urea peroxide, calcium peroxide, carbamide peroxide, magnesium peroxide, zinc peroxide, strontium peroxide and mixtures thereof. In one embodiment the peroxide compound is carbamide peroxide. Suitable metal chlorites include calcium chlorite, barium chlorite, magnesium chlorite, lithium chlorite, sodium chlorite, and potassium chlorite. Additional whitening actives may be hypochlorite and chlorine dioxide. In one embodiment the chlorite is sodium chlorite. In another embodiment the percarbonate is sodium percarbonate. In one embodiment the persulfates are oxones. The level of these substances is dependent on the available oxygen or chlorine, respectively, that the molecule is capable of providing to bleach the stain. In one embodiment the whitening agents may be present at levels from about 0.01% to about 40%, in another embodiment from about 0.1% to about 20%, in another embodiment form about 0.5% to about 10%, and in another embodiment from about 4% to about 7%, by weight of the dentifrice composition.
e) Anti-Microbial Agent

Anti-microbial agents may be included in the dentifrice compositions of the present invention. Such agents may include, but are not limited to: 5-chloro-2-(2,4-dichlorophenoxy)-phenol, commonly referred to as triclosan; 8-hydroxyquinoline and its salts; copper II compounds, including, but not limited to, copper(II) chloride, copper(II) sulfate, copper(II) acetate, copper(II) fluoride and copper(II) hydroxide; phthalic acid and its salts including, but not limited to those disclosed in U.S. Pat. No. 4,994,262, including magnesium monopotassium phthalate; chlorhexidine; alexidine; hexetidine; sanguinarine; benzalkonium chloride; salicylanilide; domiphen bromide; cetylpyridinium chloride (CPC); tetradecylpyridinium chloride (TPC); N-tetradecyl-4-ethylpyridinium chloride (TDEPC); octenidine; iodine; sulfonamides; bisbiguanides; phenolics; delmopinol, octapinol, and other piperidino derivatives; niacin preparations; zinc or stannous ion agents; nystatin; grapefruit extract; apple extract; thyme oil; thymol; antibiotics such as augmentin, amoxicillin, tetracycline, doxycycline, minocycline, metronidazole, neomycin, kanamycin, cetylpyridinium chloride, and clindamycin; analogs and salts of the above; methyl salicylate; hydrogen peroxide; metal salts of chlorite; and mixtures of all of the above. Anti-microbial components may be present from about 0.001% to about 20% by weight of the dentifrice composition. In another embodiment the antimicrobial agents generally comprise from about 0.1% to about 5% by weight of the dentifrice compositions of the present invention.
f) Anti-Plaque Agent

The dentifrice compositions of the present invention may include an anti-plaque agent such as stannous salts, copper salts, strontium salts, magnesium salts or a dimethicone copolyol. The dimethicone copolyol is selected from C12 to C20 alkyl dimethicone copolyols and mixtures thereof. In one embodiment the dimethicone copolyol is cetyl dimethicone copolyol marketed under the Trade Name Abil EM90. The dimethicone copolyol in one embodiment can be present in a level of from about 0.001% to about 25%, in another embodiment from about 0.01% to about 5%, and in another embodiment from about 0.1% to about 1.5% by weight of the dentifrice composition.
g) Anti-Inflammatory Agent

Anti-inflammatory agents can also be present in the dentifrice compositions of the present invention. Such agents may include, but are not limited to, non-steroidal anti-inflammatory (NSAID) agents oxicams, salicylates, propionic acids, acetic acids and fenamates. Such NSAIDs include but are not limited to ketorolac, flurbiprofen, ibuprofen, naproxen, indomethacin, diclofenac, etodolac, indomethacin, sulindac, tolmetin, ketoprofen, fenoprofen, piroxicam, nabumetone, aspirin, diflunisal, meclofenamate, mefenamic acid, oxyphenbutazone, phenylbutazone and acetaminophen. Use of NSAIDs such as ketorolac are claimed in U.S. Pat. No. 5,626,838. Disclosed therein are methods of preventing and/or treating primary and reoccurring squamous cell carcinoma of the oral cavity or oropharynx by topical administration to the oral cavity or oropharynx of an effective amount of an NSAID. Suitable steroidal anti-inflammatory agents include corticosteroids, such as fluccinolone, and hydrocortisone.
h) Nutrients

Nutrients may improve the condition of the oral cavity and can be included in the dentifrice compositions of the present invention. Nutrients include minerals, vitamins, oral nutritional supplements, enteral nutritional supplements, and mixtures thereof. Useful minerals include calcium, phosphorus, zinc, manganese, potassium and mixtures thereof. Vitamins can be included with minerals or used independently. Suitable vitamins include Vitamins C and D, thiamine, riboflavin, calcium pantothenate, niacin, folic acid, nicotinamide, pyridoxine, cyanocobalamin, para-aminobenzoic acid, bioflavonoids, and mixtures thereof. Oral nutritional supplements include amino acids, lipotropics, fish oil, and mixtures thereof. Amino acids include, but are not limited to L-Tryptophan, L-Lysine, Methionine, Threonine, Levocarnitine or L-carnitine and mixtures thereof. Lipotropics include, but are not limited to, choline, inositol, betaine, linoleic acid, linolenic acid, and mixtures thereof. Fish oil contains large amounts of Omega-3 (N-3) polyunsaturated fatty acids, eicosapentaenoic acid and docosahexaenoic acid. Enteral nutritional supplements include, but are not limited to, protein products, glucose polymers, corn oil, safflower oil, medium chain triglycerides. Minerals, vitamins, oral nutritional supplements and enteral nutritional supplements are described in more detail in Drug Facts and Comparisons (loose leaf drug information service), Wolters Kluer Company, St. Louis, Mo., ©1997, pps. 3-17 and 54-57.
i) Antioxidants

Antioxidants are generally recognized as useful in dentifrice compositions. Antioxidants are disclosed in texts such as Cadenas and Packer, The Handbook of Antioxidants, ©1996 by Marcel Dekker, Inc. Antioxidants useful in the present invention include, but are not limited to, Vitamin E, ascorbic acid, Uric acid, carotenoids, Vitamin A, flavonoids and polyphenols, herbal antioxidants, melatonin, aminoindoles, lipoic acids and mixtures thereof.
j) Analgesic and Anesthetic Agents

Anti-pain or desensitizing agents can also be present in the dentifrice compositions of the present invention. Analgesics are agents that relieve pain by acting centrally to elevate pain threshold without disturbing consciousness or altering other sensory modalities. Such agents may include, but are not limited to: strontium chloride; potassium nitrate; sodium fluoride; sodium nitrate; acetanilide; phenacetin; acertophan; thiorphan; spiradoline; aspirin; codeine; thebaine; levorphenol; hydromorphone; oxymorphone; phenazocine; fentanyl; buprenorphine; butaphanol; nalbuphine; pentazocine; natural herbs, such as gall nut; Asarum; Cubebin; Galanga; scutellaria; Liangmianzhen; and Baizhi. Anesthetic agents, or topical analgesics, such as acetaminophen, sodium salicylate, trolamine salicylate, lidocaine and benzocaine may also be present. These analgesic actives are described in detail in Kirk-Othmer, Encyclopedia of Chemical Technology, Fourth Edition, Volume 2, Wiley-Interscience Publishers (1992), pp. 729-737.
k) H-1 and H-2 Antagonists

The present invention may also optionally comprise selective H-1 and H-2 antagonists including compounds disclosed in U.S. Pat. No. 5,294,433.
l) Antiviral Actives

Antiviral actives useful in the present composition include any know actives that are routinely use to treat viral infections. Such anti-viral actives are disclosed in Drug Facts and Comparisons, Wolters Kluer Company, (1997, pp. 402(a)-407(z). Specific examples include anti-viral actives disclosed in U.S. Pat. No. 5,747,070, issued May 5, 1998. Said Patent discloses the use of stannous salts to control viruses. Stannous salts and other anti-viral actives are described in detail in Kirk & Othmer, Encyclopedia of Chemical Technology, Third Edition, Volume 23, Wiley-Interscience Publishers (1982), pp. 42-71. The stannous salts that may be used in the present invention would include organic stannous carboxylates and inorganic stannous halides. While stannous fluoride may be used, it is typically used only in combination with another stannous halide or one or more stannous carboxylates or another therapeutic agent.
m) Chelant

Chelating agents are able to complex calcium found in the cell walls of bacteria and can help to disrupt plaque by removing calcium from the calcium bridges which help hold this biomass intact. Suitable chelating agents include tartaric acid and salts thereof, citric acid and alkali metal citrates, soluble pyrophosphates, anionic polymeric polycarboxylates, and combinations thereof.
n) Additional actives

Additional actives suitable for use in the present invention may include, but are not limited to, insulin, steroids, herbal and other plant derived remedies. Additionally, anti-gingivitis or gum care agents known in the art may also be included. Components which impart a clean feel to the teeth may optionally be included. These components may include, for example, baking soda or Glass-H. Also, it is recognized that in certain forms of therapy, combinations of these above-named agents may be useful in order to obtain an optimal effect. Thus, for example, an anti-microbial and an anti-inflammatory agent may be combined in a single dentifrice composition to provide combined effectiveness.

Optional agents to be used include such known materials as synthetic anionic polymers, including polyacrylates and copolymers of maleic anhydride or acid and methyl vinyl ether (e.g., Gantrez), as described, for example, in U.S. Pat. No. 4,627,977, as well as, e.g., polyamino propoane sulfonic acid (AMPS), zinc citrate trihydrate, polyphosphates (e.g., tripolyphosphate; hexametaphosphate), diphosphonates (e.g., EHDP; AHP), polypeptides (such as polyaspartic and polyglutamic acids), and mixtures thereof. Additionally, the dentifrice composition can include a polymer carrier, such as those described in U.S. Pat. Nos. 6,682,722 and 6,589,512 and U.S. application Ser. Nos. 10/424,640 and 10/430,617.
o) Buffering agents

The dentifrice compositions may contain a buffering agent. Buffering agents, as used herein, refer to agents that can be used to adjust the pH of the oral compositions to a range of about pH 3.0 to about pH 10. The buffering agents include alkali metal hydroxides, ammonium hydroxide, organic ammonium compounds, carbonates, sesquicarbonates, borates, silicates, phosphates, imidazole, and mixtures thereof. Specific buffering agents include monosodium phosphate, trisodium phosphate, sodium benzoate, benzoic acid, sodium hydroxide, potassium hydroxide, alkali metal carbonate salts, sodium carbonate, imidazole, pyrophosphate salts, citric acid, and sodium citrate. Buffering agents are used at a level of from about 0.1% to about 30%, preferably from about 0.1% to about 10%, and more preferably from about 0.3% to about 3%, by weight of the oral composition.
p) Abrasive Polishing Materials

An abrasive polishing material may also be included in the oral compositions. The abrasive polishing material contemplated for use in the compositions of the present invention can be any material that does not excessively abrade dentin. Typical abrasive polishing materials include silicas including gels and precipitates; aluminas; phosphates including orthophosphates, polymetaphosphates, and pyrophosphates; and mixtures thereof. Specific examples include dicalcium orthophosphate dihydrate, calcium pyrophosphate, tricalcium phosphate, calcium polymetaphosphate, insoluble sodium polymetaphosphate, hydrated alumina, beta calcium pyrophosphate, calcium carbonate, and resinous abrasive materials such as particulate condensation products of urea and formaldehyde, and others such as disclosed by Cooley et al in U.S. Pat. No. 3,070,510, issued Dec. 25, 1962. Mixtures of abrasives may also be used. If the oral composition or particular phase comprises a polyphosphate having an average chain length of about 4 or more, calcium containing abrasives and alumina are not preferred abrasives. The most preferred abrasive is silica.

Silica dental abrasives of various types are preferred because of their unique benefits of exceptional dental cleaning and polishing performance without unduly abrading tooth enamel or dentine. The silica abrasive polishing materials herein, as well as other abrasives, generally have an average particle size ranging between about 0.1 to about 30 microns, and preferably from about 5 to about 15 microns. The abrasive can be precipitated silica or silica gels such as the silica xerogels described in Pader et al., U.S. Pat. No. 3,538,230, issued Mar. 2, 1970, and DiGiulio, U.S. Pat. No. 3,862,307, issued Jan. 21, 1975. Preferred are the silica xerogels marketed under the trade name “Syloid” by the W.R. Grace & Company, Davison Chemical Division. Also preferred are the precipitated silica materials such as those marketed by the J. M. Huber Corporation under the trade name, “Zeodent”, particularly the silica carrying the designation “Zeodent 119.” The types of silica dental abrasives useful in the toothpastes of the present invention are described in more detail in Wason, U.S. Pat. No. 4,340,583, issued Jul. 29, 1982. Silica abrasives are also described in Rice, U.S. Pat. Nos. 5,589,160; 5,603,920; 5,651,958; 5,658,553; and 5,716,601. The abrasive in the oral compositions described herein is generally present at a level of from about 6% to about 70% by weight of the composition. Preferably, oral compositions contain from about 10% to about 50% of abrasive, by weight of the oral composition.
q) Titanium dioxide may also be added to the present composition. Titanium dioxide is a white powder which adds opacity to the compositions. Titanium dioxide generally comprises from about 0.25% to about 5%, by weight of the composition.
r) Coloring agents may also be added to the present composition. The coloring agent may be in the form of an aqueous solution, preferably 1% coloring agent in a solution of water. Pigments, pealing agents, filler powders, talc, mica, magnesium carbonate, calcium carbonate, bismuth oxychloride, zinc oxide, and other materials capable of creating a visual change to the oral compositions may also be used. Color solutions and other agents generally comprise from about 0.01% to about 5%, by weight of the composition.
s) Suitable flavoring components include oil of wintergreen, clove bud oil, menthol, anethole, methyl salicylate, eucalyptol, cassia, 1-menthyl acetate, sage, eugenol, parsley oil, oxanone, alpha-irisone, marjoram, lemon, orange, propenyl guaethol, cinnamon, vanillin, ethyl vanillin, heliotropine, 4-cis-heptenal, diacetyl, methyl-para-tert-butyl phenyl acetate, cranberry, chocolate, green tea, and mixtures thereof. Coolants may also be part of the flavor composition. Coolants suitable for the present compositions include the paramenthan carboxyamide agents such as N-ethyl-p-menthan-3-carboxamide (known commercially as WS-3, WS-23, WS-5), MGA, TK-10, Physcool, and mixtures thereof. Salivating agents, warming agents, numbing agents, and other optional materials can be used to deliver a signal while the oral composition is being used. A flavor composition is generally used in the oral care compositions at levels of from about 0.001% to about 5%, by weight of the oral care composition. The flavor composition will preferably be present in an amount of from about 0.01% to about 4%, more preferably from about 0.1% to about 3%, and more preferably from about 0.5% to about 2% by weight.
t) Sweetening agents can be added to the compositions. These include saccharin, dextrose, sucrose, lactose, xylitol, maltose, levulose, aspartame, sodium cyclamate, D-tryptophan, dihydrochalcones, acesulfame, sucralose, neotame, and mixtures thereof. Various coloring agents may also be incorporated in the present invention. Sweetening agents are generally used in toothpastes at levels of from about 0.005% to about 5%, by weight of the composition.
u) Thickening agents

Additional thickening agents, such as polymeric thickeners, may be utilized. Suitable thickening agents are carboxyvinyl polymers, carrageenan, hydroxyethyl cellulose, laponite and water soluble salts of cellulose ethers such as sodium carboxymethylcellulose and sodium carboxymethyl hydroxyethyl cellulose. Natural gums such as gum karaya, xanthan gum, gum arabic, and gum tragacanth can also be used. Colloidal magnesium aluminum silicate or finely divided silica can be used as part of the thickening agent to further improve texture. Thickening agents can include polymeric polyether compounds, e.g., polyethylene or polypropylene oxide (M.W. 300 to 1,000,000), capped with alkyl or acyl groups containing 1 to about 18 carbon atoms.

A suitable class of thickening or gelling agents includes a class of homopolymers of acrylic acid crosslinked with an alkyl ether of pentaerythritol or an alkyl ether of sucrose, or carbomers. Carbomers are commercially available from B.F. Goodrich as the Carbopol® series. Particularly the carbopols include Carbopol 934, 940, 941, 956, and mixtures thereof.

Copolymers of lactide and glycolide monomers, the copolymer having the molecular weight in the range of from about 1,000 to about 120,000 (number average), are useful for delivery of actives into the periodontal pockets or around the periodontal pockets as a “subgingival gel carrier.” These polymers are described in U.S. Pat. Nos. 5,198,220; 5,242,910; and 4,443,430.

Thickening agents in an amount from about 0% to about 15%, or from about 0.01% to about 6%, in another embodiment from about 0.1% to about 5%, by weight of the total oral composition, can be used.
v) Humectant

A humectant can help to keep the dentifrice composition from hardening upon exposure to air and provide a moist feel in the mouth. A humectant or additional solvent may be added to the oral carrier phase. Suitable humectants for the present invention include water, edible polyhydric alcohols such as glycerin, sorbitol, xylitol, butylene glycol, polyethylene glycol, propylene glycol, and combinations thereof. Sorbitol, glycerin, water, and combinations thereof are preferred humectants. The humectant may be present in an amount of from about 0.1% to about 99%, from about 0.5% to about 95%, and from about 1% to about 90%.
w) Surfactants

A surfactant may be added to the dentifrice composition. Surfactants, also commonly referred to as sudsing agents, may aid in the cleaning or foaming of the oral composition. Suitable surfactants are those which are reasonably stable and foam throughout a wide pH range. The surfactant may be anionic, nonionic, amphoteric, zwitterionic, cationic, or mixtures thereof.

Examples of anionic surfactants useful herein include the water-soluble salts of alkyl sulfates having from 8 to 20 carbon atoms in the alkyl radical (e.g., sodium alkyl sulfate) and the water-soluble salts of sulfonated monoglycerides of fatty acids having from 8 to 20 carbon atoms. Sodium lauryl sulfate (SLS) and sodium coconut monoglyceride sulfonates are examples of anionic surfactants of this type. Examples of other suitable anionic surfactants are sarcosinates, such as sodium lauroyl sarcosinate, taurates, sodium lauryl sulfoacetate, sodium lauroyl isethionate, sodium laureth carboxylate, and sodium dodecyl benzenesulfonate. Mixtures of anionic surfactants can also be employed. Many suitable anionic surfactants are disclosed by Agricola et al., U.S. Pat. No. 3,959,458, issued May 25, 1976. In some embodiments, the oral composition may comprise an anionic surfactant at a level of from about 0.025% to about 9%, from about 0.05% to about 5% in some embodiments, and from about 0.1% to about 1% in other embodiments.

Another suitable surfactant is one selected from the group consisting of sarcosinate surfactants, isethionate surfactants and taurate surfactants. Preferred for use herein are alkali metal or ammonium salts of these surfactants, such as the sodium and potassium salts of the following: lauroyl sarcosinate, myristoyl sarcosinate, palmitoyl sarcosinate, stearoyl sarcosinate and oleoyl sarcosinate. The sarcosinate surfactant may be present in the compositions of the present invention from about 0.1% to about 2.5%, or from about 0.5% to about 2% by weight of the total composition.

Cationic surfactants useful in the present invention include derivatives of aliphatic quaternary ammonium compounds having one long alkyl chain containing from about 8 to 18 carbon atoms such as lauryl trimethylammonium chloride; cetyl pyridinium chloride; cetyl trimethylammonium bromide; di-isobutylphenoxyethyl-dimethylbenzylammonium chloride; coconut alkyltrimethylammonium nitrite; cetyl pyridinium fluoride; etc. Preferred compounds are the quaternary ammonium fluorides described in U.S. Pat. No. 3,535,421, Oct. 20, 1970, to Briner et al., where said quaternary ammonium fluorides have detergent properties. Certain cationic surfactants can also act as germicides in the compositions disclosed herein. Cationic surfactants such as chlorhexidine, although suitable for use in the current invention, are not preferred due to their capacity to stain the oral cavity's hard tissues. Persons skilled in the art are aware of this possibility and should incorporate cationic surfactants only with this limitation in mind.

Nonionic surfactants that can be used in the compositions of the present invention include compounds produced by the condensation of alkylene oxide groups (hydrophilic in nature) with an organic hydrophobic compound which may be aliphatic or alkylaromatic in nature. Examples of suitable nonionic surfactants include the Pluronics, polyethylene oxide condensates of alkyl phenols, products derived from the condensation of ethylene oxide with the reaction product of propylene oxide and ethylene diamine, ethylene oxide condensates of aliphatic alcohols, long chain tertiary amine oxides, long chain tertiary phosphine oxides, long chain dialkyl sulfoxides and mixtures of such materials.

Zwitterionic synthetic surfactants useful in the present invention include derivatives of aliphatic quaternary ammonium, phosphonium, and sulfonium compounds, in which the aliphatic radicals can be straight chain or branched, and wherein one of the aliphatic substituents contains from about 8 to 18 carbon atoms and one contains an anionic water-solubilizing group, e.g., carboxy, sulfonate, sulfate, phosphate or phosphonate.

Suitable betaine surfactants are disclosed in U.S. Pat. No. 5,180,577 to Polefka et al., issued Jan. 19, 1993. Typical alkyl dimethyl betaines include decyl betaine or 2-(N-decyl-N,N-dimethylammonio)acetate, coco betaine or 2-(N-coc-N,N-dimethyl ammonio)acetate, myristyl betaine, palmityl betaine, lauryl betaine, cetyl betaine, cetyl betaine, stearyl betaine, etc. The amidobetaines are exemplified by cocoamidoethyl betaine, cocoamidopropyl betaine, lauramidopropyl betaine and the like. The betaines of choice are preferably the cocoamidopropyl betaine and, more preferably, the lauramidopropyl betaine.

### Figures

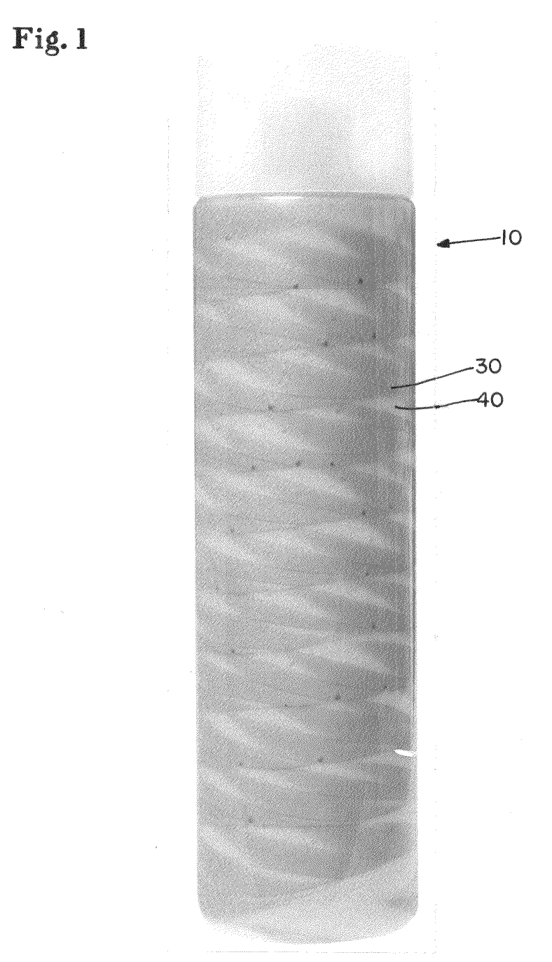

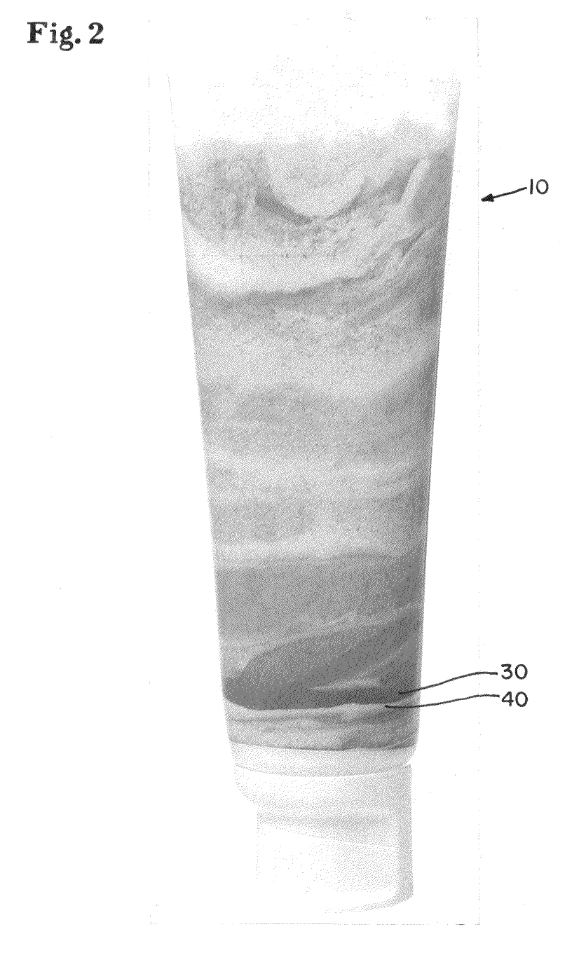

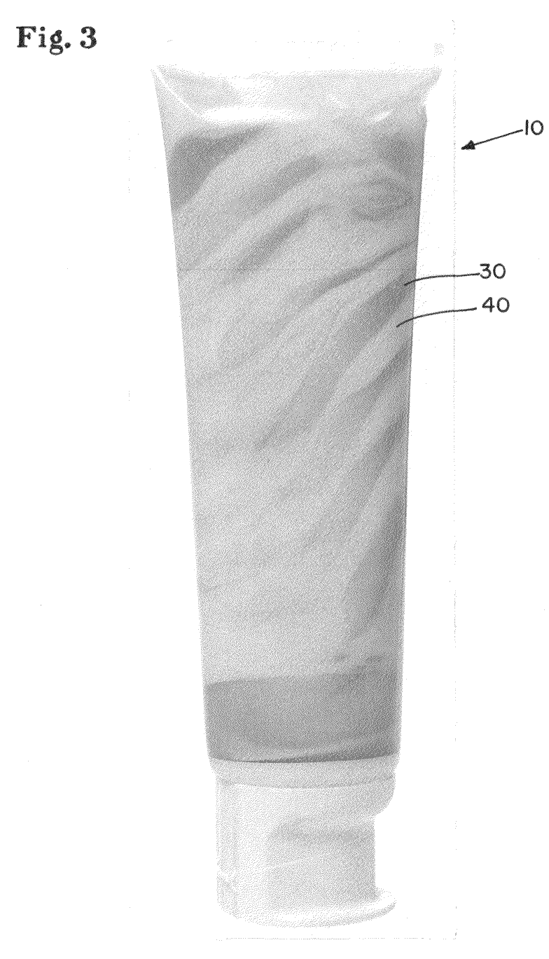

FIGS. 1-3 are photographs of three embodiments of the present invention. In FIGS. 1-3, at least two visually distinct phases are packaged in a generally transparent container 10, at least one visually distinct phase 30 is in physical contact with another phase 40, all phases are opaque, and all phases form a pattern that is not stripes. FIG. 1 shows a repeating pattern, while FIGS. 2 and 3 demonstrate marbled patterns.

The embodiment shown in FIG. 1 would suitably be produced using a filling nozzle feeding two visually distinct phases together. The phases, off-white 40 and green 30 as shown, would be volumetrically or flow-meter dosed into the filling nozzle. The filling nozzle would be internally divided for some portion of its length, separating the two phases into multiple individual streams. In this example, the diameter of the nozzle outlet would be relatively small compared to the inner diameter of the container, on the order of a 1:3 ratio. The container 10 would be raised to the fill nozzle at the start of filling process, then lowered in a controlled manner to control the distance from the filling nozzle outlet to the top level of the phases being filled. The container or filling nozzle would be moved with rotational and orbital motions during the filling process. The controlled relative motion of the nozzle and container, plus the relative pumping rates of the two phases, would suitably be achieved with servo motor technology. The container closure would suitably be placed to minimize or eliminate any air captured in the container.

The embodiments shown in FIGS. 2 and 3 would suitably be produced using a filling nozzle with a static mixer feeding two visually distinct phases together. The static mixing leads to a marbleized appearance. Both phases, 30 and 40, would be metered in a controlled manner into the fill nozzle using volumetric or flow-meter filling. The container 10 would be raised to the fill nozzle at the start of filling process, then lowered in a controlled manner to control the distance from the filling nozzle outlet to the top level of the phases being filled. The container and filling nozzle would be moved with rotational relative motion during the filling process. Variations in flow rates and rotational speeds create different pitches and horizontal contours of the patterns. The controlled relative motion of the nozzle and container, plus the relative pumping rates of the two phases, would suitably be achieved with servo motor technology. The container would suitably be designed to minimize or eliminate any air captured in the container.

### NON-LIMITING EXAMPLE

The dentifrice compositions illustrated in the following examples illustrate specific embodiments of the dentifrice compositions of the present invention, but are not intended to be limiting thereof. Other modifications can be undertaken by the skilled artisan without departing from the spirit and scope of this invention. Specifically, examples 1 and 2 are each a dentifrice with two visually distinct phases, wherein visually distinct phases I and II are opaque.

### Example 1

| | Phase I | Phase II |
|---|---|---|
| Sorbitol Solution, USP (70%, LRS) | 67.41% | 67.84% |
| PURIFIED WATER, USP, PhEur, JP, JSCI | 6.00% | 6.00% |
| Polyethylene Glycol 600 | 3.00% | 3.00% |
| CMC Sodium, USP (7M8SF-P&G) | 0.75% | 0.75% |
| Sodium Fluoride, USP | 0.24% | 0.24% |
| Saccharin Sodium, USP (Granular) | 0.25% | 0.25% |
| Titanium Dioxide, USP (Rutile) | 0.53% | 0.10% |
| Carbomer 956 | 0.30% | 0.30% |
| Sodium Phosphate, Monobasic Monohyd., USP | 0.42% | 0.42% |
| Sodium Phosphate, Tribasic, Dodecahyd., FCC | 1.10% | 1.10% |
| Silica, Dent Type (7% LOD)(Zeodent 119) | 15.00% | 15.00% |
| Sodium Lauryl Sulfate (28% solution) | 4.00% | 4.00% |
| Flavor | 0.80% | 0.80% |
| Sorbosil BFG52 | 0.20% | |

### Example 2

| | Phase I | Phase II |
|---|---|---|
| Sorbitol Solution, USP (LRS) | 31.62% | 25.24% |
| Sodium Monofluorophosphate | 0.76% | 0.76% |
| Usp Water | 34.00% | 19.00% |
| Polyethylene Glycol 600, NF | 3.00% | 3.00% |
| Sodium Acid Pyrophosphate FCC Anhydrous | 4.17% | 0.20% |
| Carbomer 956 | 0.40% | |
| Saccharin Sodium, USP | 0.35% | 0.30% |
| Xanthan Gum, NF | 0.70% | |
| Carboxymethyl Cellulose | | 1.50% |
| Sodium Hydroxide Solution 50% FCC | 3.50% | |
| Silica, Dental Type, NF (Zeodent 119) | 15.00% | |
| Calcium Carbonate | | 42.00% |
| Flavor | 1.00% | 1.00% |
| Sodium Lauryl Sulfate 28% Solution | 5.00% | 7.00% |
| Dye (1% sol'n) | 0.30% | |
| Sorbosil BFG52 | 0.20% | |

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this written document conflicts with any meaning or definition of the term in a document incorporated by reference, the meaning or definition assigned to the term in this written document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-phase dentifrice composition comprising: at least two visually distinct phases;
wherein said visually distinct phases are packaged in a generally transparent container;
wherein at least one visually distinct phase is in physical contact with another phase;
wherein all said visually distinct phases are opaque; and
wherein said visually distinct phases form a pattern that is not striped.

2. The composition of claim 1, wherein said pattern is selected from the group consisting of marbled, geometric, starburst, lightning, blocks, and combinations thereof.

3. The composition of claim 1, wherein at least one phase is pearlescent.

4. The composition of claim 1, wherein at least one phase is photosensitive.

5. The composition of claim 1, wherein at least one phase contains suspended particles.

6. The composition of claim 1, wherein each visually distinct phase comprises at least 10% of the volume of all phases combined.

7. The composition of claim 1, wherein at least one phase forms a pattern, said pattern selected from the group consisting of textured, pleated, cupped, concave, convex, braided, and tapered.

8. The composition of claim 1, wherein at least one phase appears textured.

9. The composition of claim 1, wherein at least one phase appears pleated.

10. The composition of claim 1, wherein the container is patterned, said pattern selected from the group consisting of striped, marbled, spiral, geometric, starburst, lightning, blocks, colored, tinted, shaded, frosted, and combinations thereof.

11. The composition of claim 1, wherein said visually distinct phases are further packaged in at least one packaging layer selected from the group consisting of a label, shrink-wrap, a box, and combinations thereof; and
wherein at least one packaging layer is patterned, said pattern selected from the group consisting of striped, marbled, spiral, geometric, starburst, lightning, blocks, colored, tinted, shaded, frosted, and combinations thereof.

12. The composition of claim 11, wherein the combination of the dentifrice and container and/or packaging layer form a pattern.

13. The composition of claim 1, wherein said container includes a visible strip inside the container.

14. The composition of claim 1, wherein at least one phase comprises a benefit phase.

15. A method of forming a multi-phase dentifrice according to claim 1, comprising the steps of:
providing at least two visually distinct dentifrice phases, each stored in a storage bin having a pump and a hose attached thereto;
moving a container for receiving said multi-phase dentifrice into a position relative to a support and alignment funnel;
pumping the at least two visually distinct phases through the respective hoses into a nozzle assembly having at least two nozzles for filling the container; and
combining predetermined amounts of each of the at least two visually distinct phases for creating a multi-phase dentifrice in a single container.

16. The method of claim 15, wherein the filling of at least one phase into said container is continuous.

17. The method of claim 15, wherein the filling of at least one phase into said container is discontinuous.

18. The method of claim 15, wherein at least one nozzle engages in motion during filling of the container; and
wherein the motion is selected from the group consisting of oscillating, reciprocating, translating, vibrating, pulsating, rotating, plunging, and combinations thereof.

19. The method of claim 15, wherein said container engages in motion during filling of the container,
wherein the motion is selected from the group consisting of oscillating, reciprocating, translating, vibrating, pulsating, rotating, plunging, and combinations thereof.

20. The method of claim 15, wherein the appearance formed by the visually distinct phases after filling the container is achieved by an action selected from the group consisting of centrifuging the container, shaking the container, changing the temperature of the container, changing the pressure of the container, adding air to the container, removing air from the container, applying electromagnetic radiation, and applying sonic energy.
